(19) United States
(12) Patent Application Publication
(10) Pub. No.: US 2015/0202161 A1
(43) Pub. Date: Jul. 23, 2015

(54) PROCESS FOR MANUFACTURING DRUG DELIVERY FORMULATIONS

(71) Applicant: O-RAY PHARMA, INC., Pasadena, CA (US)

(72) Inventors: Erik Pierstorff, Sierra Madre, CA (US); William H. Slattery, La Canada, CA (US)

(73) Assignee: O-RAY PHARMA, INC., Pasadena, CA (US)

(21) Appl. No.: 14/418,690

(22) PCT Filed: Aug. 20, 2013
(86) PCT No.: PCT/US13/55846

§ 371 Date: Jan. 30, 2015

Related U.S. Application Data
(60) Provisional application No. 61/691,178, filed on Aug. 20, 2012.

(51) Int. Cl. A61K 9/48; C07K 16/18; A61K 47/34; A61K 47/38; A61J 3/00; A61K 31/7036; C07K 16/24

(52) U.S. Cl. CPC A61K 9/4808; A61K 9/4816; A61K 31/7036; C07K 16/18; C07K 16/241; A61K 47/38; A61J 3/00; A61K 9/4891; A61K 9/4833; A61K 47/34

(57) ABSTRACT

The present invention provides for methods of producing a drug product that is capable of providing controlled and sustained release of a drug. Particularly, the release of the drug will be to a specific tissue area in the body. The methods include, but are not limited to, providing an impermeable casing with an open end, placing a compressed drug pellet into the impermeable casing, coating the open end of the impermeable casing with a permeable layer to create a release window for the drug product to provide controlled and/or sustained release of the drug.

### FIELD OF INVENTION

This invention relates to processes for manufacturing drug delivery product.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Meniere's disease is a disorder of the inner ear that causes spontaneous episodes of vertigo along with fluctuating hearing loss, ringing in the ear (tinnitus), and sometimes a feeling of fullness or pressure in the ear (aural fullness). No cure currently exists for Meniere's disease, but a number of treatment options may help manage symptoms.

Treatment options include middle ear injections where medications are injected into the middle ear, and then absorbed into the inner ear to improve vertigo symptoms. For example, gentamicin, an antibiotic that is toxic to inner ear, reduces the balancing function of the ear, and the other ear assumes responsibility for balance. The procedure, which can be performed during local anesthesia in a doctor's office, can often reduce the frequency and severity of vertigo attacks. Steroids, such as dexamethasone, also may help control vertigo attacks in some individuals. This procedure can also be performed with local anesthesia applied by a doctor. Although dexamethasone may be slightly less effective than gentamicin, dexamethasone is less likely than gentamicin to cause further hearing loss.

Various drug products have been developed to assist in the treatment of various disease conditions. However, in many instances, there are drugs that are not suitable of being administered orally or intravenously without the risks of unfavorable side effects. Even drugs that can be administered orally or intravenously may cause unwanted side effects. Additionally, for drugs that can be administered via injection, for example in the case of Meniere's disease, multiple administrations can be required to achieve the desired results.

The methods of manufacturing these drugs in the prior art frequently includes an impermeable shell packed with powdered drug, or a matrix of polymer and drug to create a drug core. These methods may not allow for reproducible load of drugs, enhanced extended release, enhanced packing, allow for a wider range of polymers to be used in manufacture and hence enhancing the extended release characteristics of the final product, or enhanced speed in manufacture. As such there remains an unmet need in the art for a drug product to provide controlled and/or sustained release of a drug to a targeted area. The methods described in the present invention provide a solution to these problems.

### SUMMARY OF THE INVENTION

The following embodiments and aspects thereof are described and illustrated in conjunction with compositions and methods which are meant to be exemplary and illustrative, not limiting in scope.

Various embodiments of the present invention provides for a method of producing a drug product, comprising: providing an impermeable casing comprising: a sealed end, a tube, and an open end; placing one or more compressed drug pellets into the impermeable casing; and coating the open end with a permeable polymer coating to produce a release window to control the release of the drug.

In various embodiments, the impermeable casing can comprise a polymer or a co-polymer comprising at least one monomer selected from the group consisting of a sugar phosphate, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene, halogenated para-xylene, β-phenol lactic acid, silicone, ethylene vinyl acetate, polyvinyl alcohol and combinations thereof. In certain embodiments, the impermeable casing can comprise parylene.

In various embodiments, the drug can be selected from the group consisting of anti-inflammatory agent, analgesic agent, corticosteroid, growth factor, antioxidant, TNF-α inhibitor, volume expanding agent, vasodilating agent, antihistaminic agent, anticholinergic agent, antibiotic agent, antiviral agent, immunosuppressive agent, diuretic agent, antacid, H2-blocker, antiemetic, calcium channel blocker, anticancer agent, vitamin, vascular rheologic agent, neuroprotective agent, neuromodulator, and anti-apoptotic agent. In certain embodiments, the drug can be fentanyl citrate, aspirin, salicylate, ibuprofen, naproxen, droperidol, prochlorperazine, dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, adalimumab, batahistine, niacin, papaverine, meclizine, dimenhydrinate, scopolamene, promethazine, glycopyrrolate, propantheline, atropine, ampicillin, cefuroxime, ceftriaxone, ciprofloxacin, finafloxacin, gatifloxacin, levofloxacin, moxifloxacin, ofloxacin, gentamicin, tobramycin, clindamycin, amoxicillin, cyclophosphamide, cyclosporine, thiazide, triamterene, nizatidine, cimetidine, metoclopramide, diphenidol, diltiazem, nifedipine, or verapamil. In various embodiments, the drug can be dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, or adalimumab. In certain embodiments, the drug can be gentamicin sulfate. In certain embodiments, the drug can be immunoglobulin G. In certain embodiments, the drug can be infliximab.

In various embodiments, the permeable polymer coating can be 5 nanometers to 50 microns thick, and the permeable polymer is a polymer or a co-polymer comprising at least one monomer selected from the group consisting of a sugar phosphate, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene, halogenated para-xylene, β-phenol lactic acid, silicone, ethylene vinyl acetate, polyvinyl alcohol and combinations thereof. In various embodiments, the permeable polymer coating can be 5 nanometers to 50 microns thick, and the permeable polymer is selected from the group consisting of parylene, polylactic acid, polyvinyl alcohol and combinations thereof. In certain embodiments, the permeable polymer coating can be less than one micron thick and the permeable polymer is parylene.

Various embodiments of the present invention provides for a method of producing a drug product, comprising: providing one or more compressed drug pellets; depositing an impermeable coating layer on the one or more drug pellets to produce a coated drug pellet; cutting a first end of the of the coated drug pellet to create an open end; coating the open end with a permeable layer of a polymer to create a release window.

In various embodiments, the compressed drug pellet can comprise a drug, a polymer and/or an excipient.

In various embodiments, two or more drug pellets can be provided and the method can further comprise joining the two or more drug pellets with a connecting substrate between the two or more drug pellets to produce a connected drug pellet. In various embodiments, the connecting substrate can be selected from the group consisting of poly lactic acid, polyvinyl alcohol, polyethylene glycol, microcrystalline cellulose and combinations thereof. In various embodiments, cutting the first end can comprise cutting the connected drug pellet through the connecting substrate to form a first open end on a first coated drug pellet and a second open end on the second coated drug pellet.

In various embodiments, the impermeable coating layer can comprise a polymer or a co-polymer comprising at least one monomer selected from the group consisting of a sugar phosphate, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene, halogenated para-xylene, β-phenol lactic acid, silicone, ethylene vinyl acetate, polyvinyl alcohol and combinations thereof. In certain embodiments, the impermeable coating layer comprises parylene and is one micron or more in thickness.

In various embodiments, the drug can be selected from the group consisting of anti-inflammatory agent, analgesic agent, corticosteroid, growth factor, antioxidant, TNF-α inhibitor, volume expanding agent, vasodilating agent, antihistaminic agent, anticholinergic agent, antibiotic agent, antiviral agent, immunosuppressive agent, diuretic agent, antacid, H2-blocker, antiemetic, calcium channel blocker, anticancer agent, vitamin, vascular rheologic agent, neuroprotective agent, neuromodulator, and anti-apoptotic agent.

In various embodiments, drug can be fentanyl citrate, aspirin, salicylate, ibuprofen, naproxen, droperidol, prochlorperazine, dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, adalimumab, batahistine, niacin, papaverine, meclizine, dimenhydrinate, scopolamene, promethazine, glycopyrrolate, propantheline, atropine, ampicillin, cefuroxime, ceftriaxone, ciprofloxacin, finafloxacin, gatifloxacin, levofloxacin, moxifloxacin, ofloxacin, gentamicin, tobramycin, clindamycin, amoxicillin, cyclophosphamide, cyclosporine, thiazide, triamterene, nizatidine, cimetidine, metoclopramide, diphenidol, diltiazem, nifedipine, or verapamil. In various embodiments, the drug can be dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, or adalimumab. In various embodiments, the drug can be gentamicin sulfate. In various embodiments, the drug can be immunoglobulin G. In various embodiments, the drug can be infliximab.

In various embodiments, the permeable layer of a polymer can be 5 nanometers to 50 microns thick, and the permeable polymer can be a polymer or a co-polymer comprising at least one monomer selected from the group consisting of a sugar phosphate, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene, halogenated para-xylene, β-phenol lactic acid, silicone, ethylene vinyl acetate, polyvinyl alcohol and combinations thereof.

In various embodiments, the permeable polymer coating can be 5 nanometers to 50 microns thick, and the permeable polymer is selected from the group consisting of parylene, polylactic acid, polyvinyl alcohol and combinations thereof. In certain embodiments, the permeable polymer layer can be less than one micron thick and the permeable polymer can be parylene.

Various embodiments of the present invention provide for a method of producing a drug product, comprising: providing a compressed drug pellet; and coating the compressed drug pellet with a biodegradable permeable polymer.

In various embodiments, the drug can be selected from the group consisting of anti-inflammatory agent, analgesic agent, corticosteroid, growth factor, antioxidant, TNF-α inhibitor, volume expanding agent, vasodilating agent, antihistaminic agent, anticholinergic agent, antibiotic agent, antiviral agent, immunosuppressive agent, diuretic agent, antacid, H2-blocker, antiemetic, calcium channel blocker, anticancer agent, vitamin, vascular rheologic agent, neuroprotective agent, neuromodulator, and anti-apoptotic agent.

In various embodiments, the drug can be fentanyl citrate, aspirin, salicylate, ibuprofen, naproxen, droperidol, prochlorperazine, dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, adalimumab, batahistine, niacin, papaverine, meclizine, dimenhydrinate, scopolamene, promethazine, glycopyrrolate, propantheline, atropine, ampicillin, cefuroxime, ceftriaxone, ciprofloxacin, finafloxacin, gatifloxacin, levofloxacin, moxifloxacin, ofloxacin, gentamicin, tobramycin, clindamycin, amoxicillin, cyclophosphamide, cyclosporine, thiazide, triamterene, nizatidine, cimetidine, metoclopramide, diphenidol, diltiazem, nifedipine, or verapamil. In various embodiments, the drug can be dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, or adalimumab. In various embodiments, the drug can be gentamicin sulfate. In various embodiments, the drug can be immunoglobulin G. In various embodiments, the drug can be infliximab.

In various embodiments, the biodegradable permeable polymer can be selected from a polymer or co-polymer including at least one monomer selected from the group consisting of sugar phosphates, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, β-phenol lactic acid, and combinations thereof.

Various embodiments of the present invention provide for a method of inhibiting, alleviating or treating a disease condition, comprising: providing a drug product of the present invention; and administering the drug product to a mammalian subject in need thereof.

In various embodiments, the disease condition can be Meniere's disease. In various embodiments, the disease condition can be ototoxicity, sensorineural hearing loss, inflammation, autoimmune inner ear disease, noise induced hearing loss, infection, or inner ear vestibular dis-function.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a method of producing a drug product in accordance with various embodiments of the present invention.

FIG. 2 depicts another method of producing a drug product in accordance with various embodiments of the present invention.

FIG. 3 depicts another method of producing a drug product in accordance with various embodiments of the present invention.

FIG. 4 depicts another method of producing a drug product in accordance with various embodiments of the present invention.

FIG. 5 depicts another method of producing a drug product in accordance with various embodiments of the present invention.

FIG. 6 depicts in vitro release of gentamicin in accordance with various embodiments of the present invention. The drug product was produced via the method depicted in FIG. 1.

FIG. 7 depicts in vivo release of gentamicin in accordance with various embodiments of the present invention. The drug product was produced via the method depicted in FIG. 1.

FIG. 8 depicts in vitro release of Immunoglobulin G in accordance with various embodiments of the present invention.

FIG. 9 depicts in vitro release of Infliximab in accordance with various embodiments of the present invention.

FIG. 10 depicts in vitro release of gentamicin in accordance with various embodiments of the present invention. The drug product was produced via the method depicted in FIG. 2.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Impermeable” as used herein refers a material through which substances, such as liquids and small molecules cannot pass. For example, materials that are impermeable will not be permeable to molecules that are about 200 to 200,000 Daltons.

“Permeable” as used herein with respect to a material refers to material that can be permeated or penetrated, especially by liquids or dissolved active ingredients in solution. For example, material that are permeable can be permeable to molecules that about 200 to 200,000 Daltons; although, different permeable materials will be permeable to molecules with different molecular weights. For example, in various embodiments, the permeable material can be permeable to molecules that are up to 200, 300, 400, 500, 1,000, 2,500, 5,000, 7,500, 10,000, 15,000, 20,000, 30,000, 40,000, 50,000, 60,000, 70,000, 80,000, 90,000, 100,000, 110,000, 120,000, 130,000 140,000 150,000 160,000 170,000 180,000 190,000, or 200,000 Daltons.

Various embodiments of the present invention provide for methods of producing a drug product. The drug product provides controlled release of the drug into a subject or a specific area of the subject in need of the drug to inhibit, alleviate or treat certain disease conditions.

In various embodiments, the drug product made by the methods of the present invention has a diameter of about 0.4 mm to about 2.0 mm. In various embodiments, the diameter is about 0.4 to 0.5, 0.5 to 0.6, 0.6 to 0.7, 0.7 to 0.8, 0.8 to 0.9, 0.9 to 1.0, 1.0 to 1.5, or 1.5 to 2.0 mm.

In various embodiments, the drug product made by the methods of the present invention has a length of about 0.5 mm to about 6.0 mm. In various embodiments, the length is about 0.5 to 0.6, 0.6 to 0.7, 0.7 to 0.8, 0.8 to 0.9, 0.9 to 1.0, 1.0 to 1.5, 1.5 to 2.0, 2.0 to 3.0, 3.0 to 4.0, 4.0 to 5.0, or 5.0 to 6.0 mm.

The thickness of the impermeable layer/coating or the impermeable casing will be dependent on the polymer used. In various embodiments, this can range from 5 to 150 microns. In certain embodiments, this can range from 5-10, 10-15, 15-20, 20-25, 25-50, 50-100, or 100-150 microns.

The thickness of the permeable layer/coating over the release window is dependent on the polymer used. In various embodiments, this can range from 5 nanometers to 50 microns. In certain embodiments, this can range from 5-10, 10-15, 15-20, 20-25, 25-50, 50-75, 75-100, 100-200, 200-300, 300-400, 400-500, 500-600, 600-700, 700-800, 800-900, or 900-1000 nanometers. In certain embodiments, this can range from 1-2, 2-3, 3-4, 4-5, 5-6, 6-7, 7-8, 8-9, 9-10, 10-15, 15-20, 20-25, 25-30, 30-35, 35-40, 40-45, or 45-50 microns.

The thickness of the permeable layer/coating around the entire drug pellet is dependent on the polymer used. This can range from 5 nanometers to 50 microns. In certain embodiments, this can range from 5-10, 10-15, 15-20, 20-25, 25-50, 50-75, 75-100, 100-200, 200-300, 300-400, 400-500, 500-600, 600-700, 700-800, 800-900, or 900-1000 nanometers. In certain embodiments, this can range from 1-2, 2-3, 3-4, 4-5, 5-6, 6-7, 7-8, 8-9, 9-10, 10-15, 15-20, 20-25, 25-30, 30-35, 35-40, 40-45, or 45-50 microns.

In various embodiments, the degree of permeability (e.g., permeability to certain molecular weights) for the permeable layer can vary to suit the needs of the drug.

In various embodiments, the drug product made by the methods of the present invention comprises gentamicin sulfate. Other embodiments of the present invention include one or more pharmacologically active compounds in the following classes of agents: anti-inflammatory and analgesic agents, including but not limited to fentanyl citrate and aspirin; non-steroidal anti-inflammatory (NSAID) agents, including but not limited to salicylates, ibuprofen, naproxen; tranquilizing agents, including but not limited to droperidol and prochlorperazine; corticosteroids, including but not limited to dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone; growth factors, including but not limited to IGF-1, FGF-2, BDNF; antioxidants, including but not limited to reduced glutathione, N-methyl-(D)-glucaminedithiocarbamate and (D)-methionine; TNF-α inhibitors, including but not limited to infliximab, etanercept, adalimumab; volume expanding agents; vasodilating agents, including but not limited to batahistine, niacin and papaverine; antihistaminic agents, including but not limited to meclizine, dimenhydrinate, scopolamene, and promethazine; anticholinergic agents, including but not limited to glycopyrrolate, propantheline, and atropine; antibiotic agents, including but not limited to ampicillin, cefuroxime, ceftriaxone, ciprofloxacin, finafloxacin, gatifloxacin, levofloxacin, moxifloxacin, ofloxacin, gentamicin, tobramycin, clindamycin, amoxicillin; antiviral agents; immunosuppressive agents, including but not limited to cyclophosphomide and cyclosporine; diuretic agents, including but not limited to thiazide, triamterene and carbonic anhydrase inhibitors; antacids and H2-blockers, including but not limited to nizatidine and cimetidine; antiemetics, including but not limited to metoclopramide or diphenidol; calcium channel blockers, including but not limited to diltiazem, nifedipine and verapamil; anticancer agents and drugs; vitamins; vascular rheologic agents; neuroprotective agents; neuromodulators; and anti-apoptotic agents.

In various embodiments, the drug product made by the methods of the present invention is configured for surgical implantation in the middle ear with delivery surface to the round window, into mucosa of middle ear, into oval window, or into the stapes. The methods include positioning a sustained released drug delivery system at an area wherein release of the agent is desired and allowing the agent to pass through the device to the desired area of treatment. Accordingly, an aspect of the invention is a method of treating a condition of the ear of a mammal comprising the steps of accessing an internal anatomical site adjacent to the inner ear, and placing or implanting a drug delivery device in the internal anatomical site. As such, the drug product made by these methods is designed to fit in the anatomical site adjacent to the inner ear, with a length and diameter as described above.

In various embodiments, the drug product made by the methods of the present invention is used for the treatment of Meniere's disease. In other embodiments, the drug product made by the methods of the present invention is used for protection against ototoxicity, prevention or reduction of the likelihood of sensorineural hearing loss, treatment of sensorineural hearing loss, protection against inflammation, treatment of autoimmune inner ear disease, prevention or reduction of the likelihood of noise induced hearing loss, treatment of noise induced hearing loss, treatment of infection, and treatment of inner ear vestibular dis-function.

In various embodiments, the drug product made by the methods of the present invention comprises about 5 μg to about 20 mg of the drug. In various embodiments, the drug product made by the methods of the present invention comprises about 5 to 10, 10 to 20, 20 to 40, 40 to 60, 60 to 80, or 80 to 100 μg of the drug. In various embodiments, the drug product made by the methods of the present invention comprises about 100 to 200, 200 to 300, 300 to 400, or 400 to 500 μg of the drug. In various embodiments, the drug product made by the methods of the present invention comprises about 0.5 to 1, 1 to 2, 2 to 3, 3 to 4, 4 to 5, 5 to 6, 6 to 7, 7 to 8, 8 to 9, or 9 to 10 mg of the drug. In various embodiments, the drug product made by the methods of the present invention comprises about 10 to 15, 15 to 20, or 20 to 25 mg of the drug.

In various embodiments, the drug product made by the methods of the present invention is configured to release the drug for a duration of about 1 day to about 6 months. In various embodiments, the duration is about 1 to 2, 2 to 3, 3 to 4, 4 to 5, 5 to 6, 6 to 7 days. In various embodiments the duration is about 1 to 2, 2 to 3, 3 to 4 weeks. In various embodiments, the duration is about 4 to 5, 5 to 6, 6 to 7, or 7 to 8 weeks. In various embodiments, the duration is about 8 to 10, 10 to 12, 12 to 14, 14 to 16, 16 to 18, 18 to 20, 20 to 22, or 22 to 24 weeks. In various embodiments, the duration is about 1 to 2, 2 to 3, 3 to 4, 4 to 5, or 5 to 6 months.

In various embodiments, the method of producing a drug product comprises: providing an impermeable casing, comprising: a sealed end, a tube (e.g., cylindrical tube), and an open end; placing one or more compressed drug pellets into the impermeable casing; and coating the open end with a permeable polymer coating to produce a release window to control the release of the drug. In various embodiments, the method further comprises compressing a drug into a compressed drug pellet.

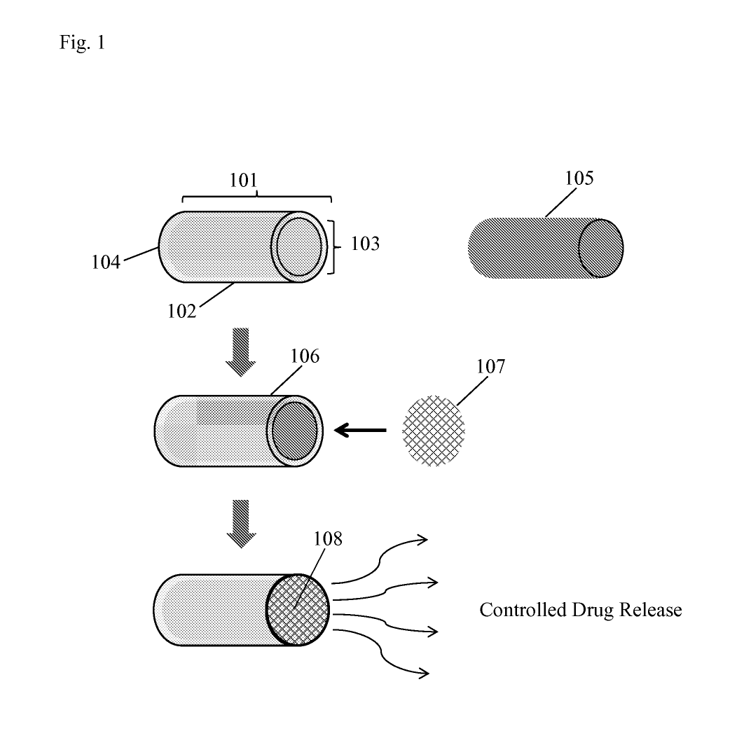

In an embodiment, FIG. 1 depicts an impermeable casing 101 comprising: a tube 102 with an open end 103 and a sealed end 104. A drug pellet 105 was formed and dip coated in, for example, polylactic acid. One or more dip-coated drug pellets were placed inside the impermeable casing (106). The open end was coated by a permeable polymer 107 to create a release window 108, which provided controlled drug release.

In various embodiments, the impermeable tube (e.g., cylindrical tube) is made of an impermeable polymer. In various embodiments, the impermeable polymer is selected from the group consisting of a polymer or co-polymer including at least one monomer selected from the group consisting of sugar phosphates, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene (parylene N), halogenated para-xylene (i.e. paylene C, parylene HT), β-phenol lactic acid, silicone, ethylene vinyl acetate, polyvinyl alcohol and combinations thereof.

In various embodiments, the drug in the compressed drug pellet is a drug as discussed herein. In various embodiments, the drug is in an amount as discussed herein.

In various embodiments, the permeable coating is selected from the group consisting of a polymer or co-polymer including at least one monomer selected from the group consisting of sugar phosphates, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene (parylene N), halogenated para-xylene (i.e. paylene C, parylene HT), β-phenol lactic acid, silicone, ethylene vinyl acetate, polyvinyl alcohol and combinations thereof. The thickness of the permeable layer over the release window is dependent on the polymer used. This can range from 5 nanometers to 50 microns. In various embodiments, the degree of permeability (e.g., permeability to certain molecular weights) for the permeable layer can vary to suit the needs of the drug.

In various embodiments, the method of producing a drug product comprises: providing a compressed drug pellet; depositing an impermeable coating layer on the drug pellet via vapor deposition; cutting a first end of the coated drug pellet to create an opened end; coating the opened end with a permeable layer of a polymer to create a release window. In various embodiments, the method further comprises compressing a drug to form a compressed drug pellet.

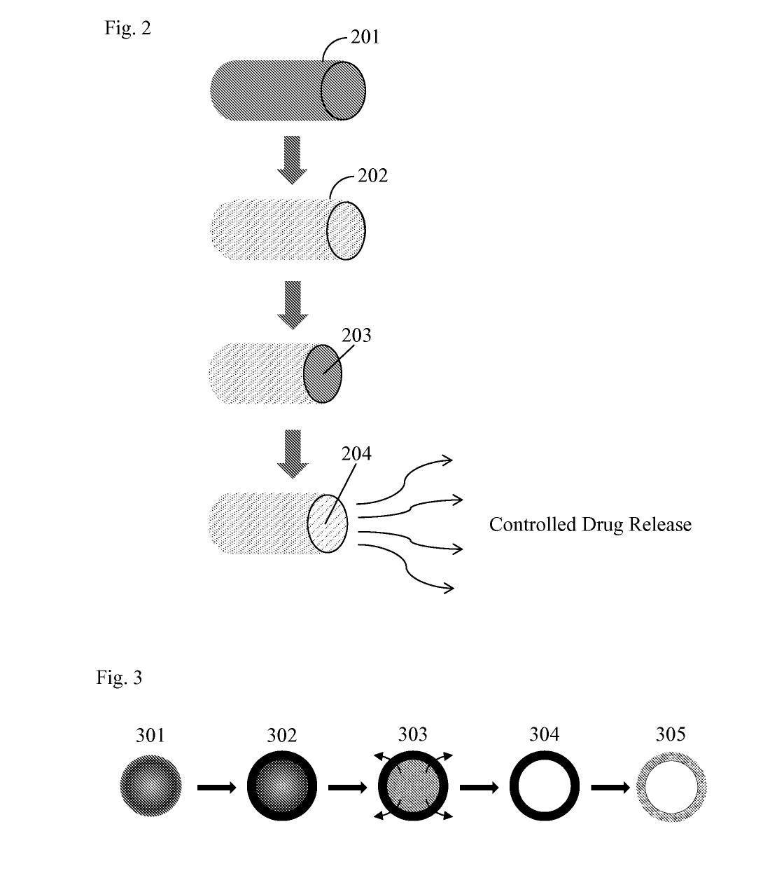

In an embodiment, FIG. 2 depicts a compressed drug pellet 201; depositing an impermeable coating layer on the drug pellet via vapor deposition 202; cutting a first end of the coated drug pellet to create an opened end 203; coating the opened end with a permeable layer of a polymer to create a release window 204.

In various embodiments, the thickness of the permeable layer of the polymer correlates to the permeability of the permeable layer and affects the release of the drug pellet. In various embodiments, the drug in the compressed drug pellet is a drug as discussed herein. In various embodiments, the drug is in an amount as discussed herein.

In various embodiments, the permeable polymer coating is selected from the group consisting of sugar phosphates, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene (parylene N), halogenated para-xylene (i.e. paraylene C, parylene HT), β-phenol lactic acid, silicone, ethylene vinyl acetate, polyvinyl alcohol and combinations thereof. In various embodiments, the degree of permeability (e.g., permeability to certain molecular weights) for the permeable layer can vary to suit the needs of the drug.

In alternative embodiments, the method of producing a drug product comprises: providing a compressed drug pellet comprising a drug, a polymer and/or an excipient; coating the drug pellet with an impermeable layer to produce a coated drug pellet; cutting one end of the coated drug pellet to create an open end; coating the opened end with a permeable layer to create a release window. In various embodiments, the method further comprises providing the drug, the polymer and/or the excipient and mixing the drug, polymer and/or excipient. In various embodiments, the method further comprises compressing a mixture of the drug, polymer and/or excipient to produce the compressed drug pellet.

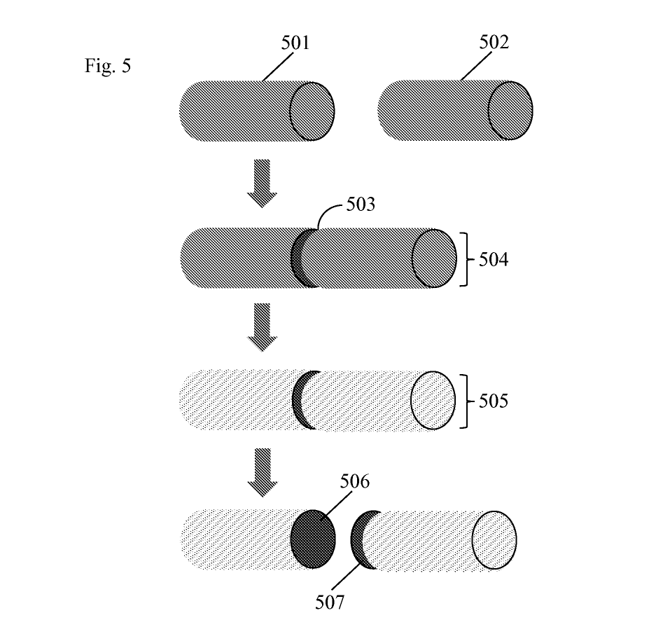

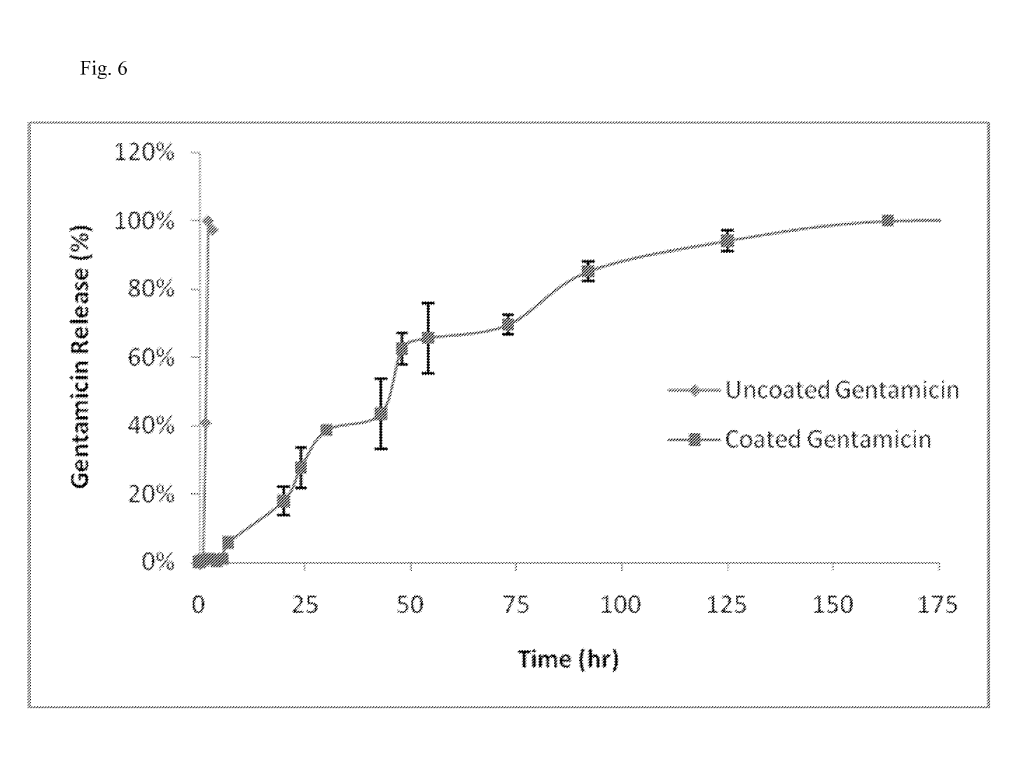

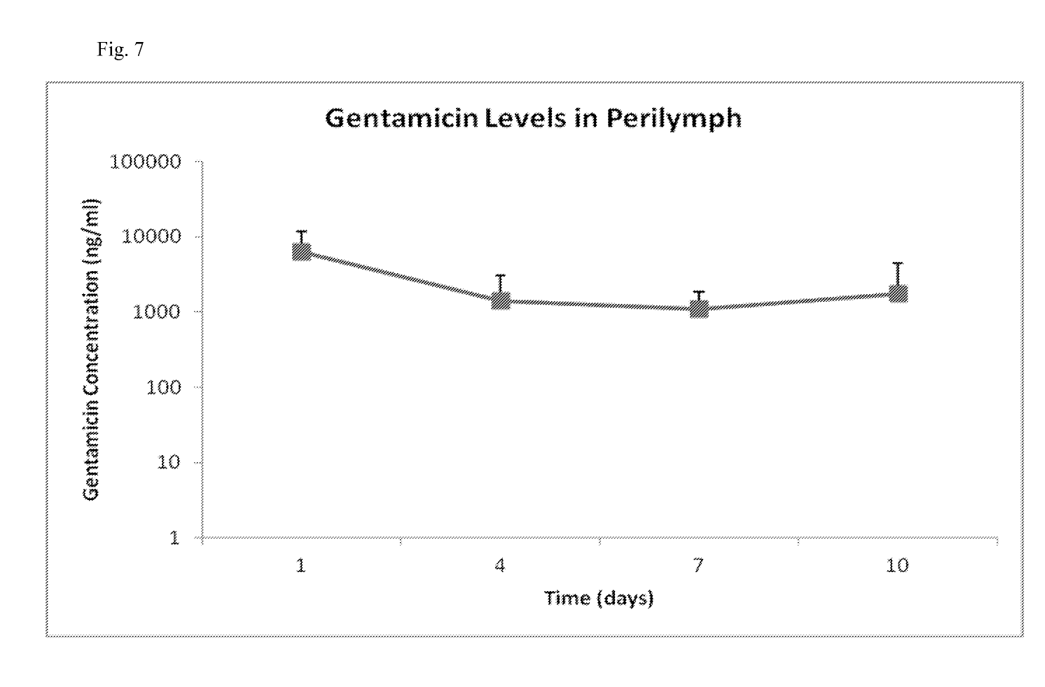

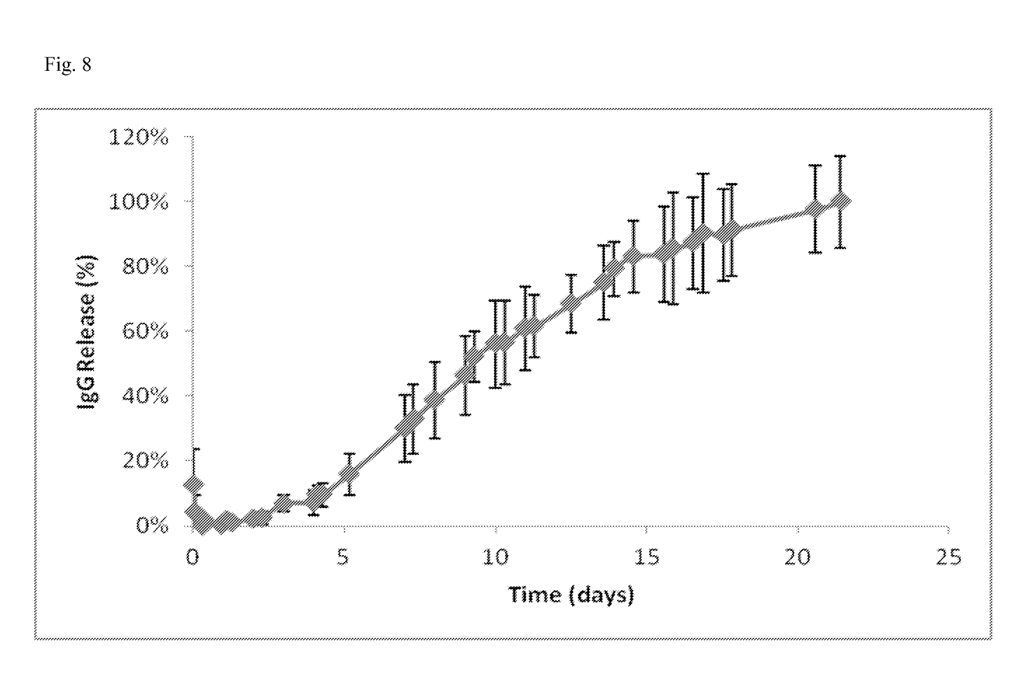

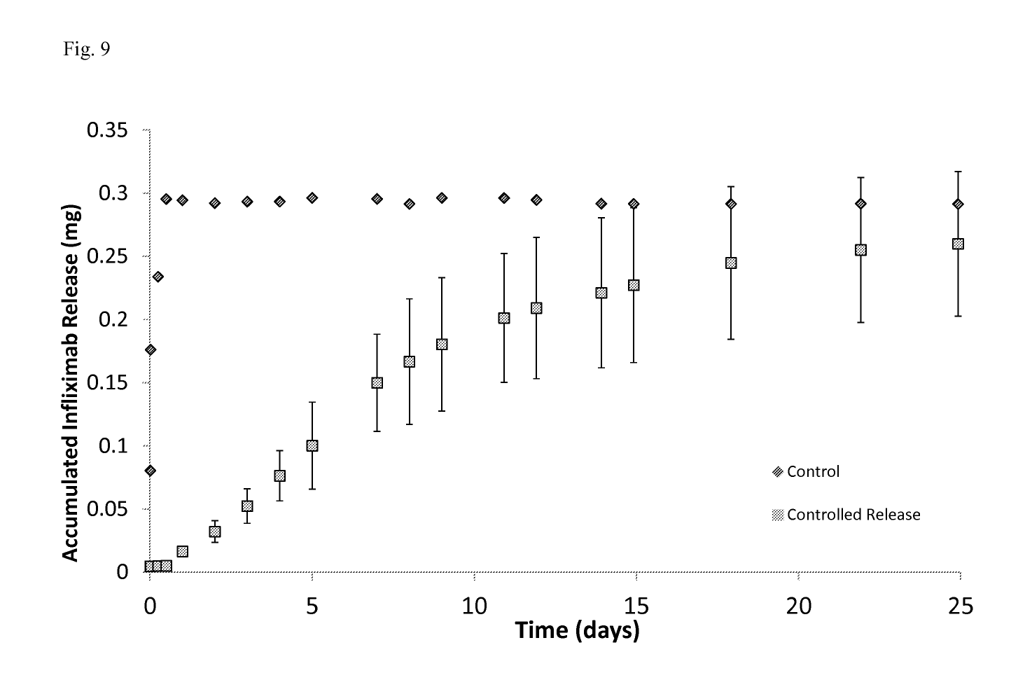

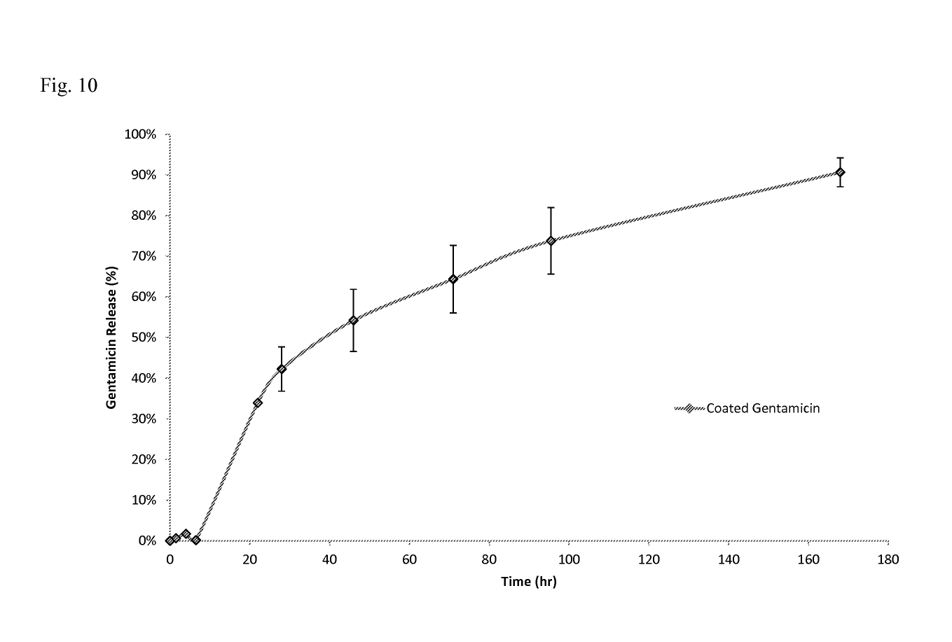

In an embodiment, FIG. 4 depicts compressing a mixture 401 of the drug, polymer and/or excipient to produce a compressed drug pellet 402. The compressed drug pellet 402 can be packed into the impermeable casing as depicted in FIG. 1. Alternatively, the compressed drug pellet can be coated with an impermeable layer to produce a coated drug pellet 403. A first end of the compressed drug pellet can be cut to create an open end 404. The open end 404 can be coated as depicted in FIG. 2.

In various embodiments, the thickness of the permeable layer of the polymer correlates to the permeability of the permeable layer and affects the release of the drug pellet. In various embodiments, the degree of permeability (e.g., permeability to certain molecular weights) for the permeable layer can vary to suit the needs of the drug.

In various embodiments, the drug in the compressed drug pellet is a drug as discussed herein. In various embodiments, the drug is in an amount as discussed herein.

In various embodiments, the polymer or excipient is selected from the group consisting of zinc carbonate, magnesium carbonate, calcium carbonate, magnesium hydroxide, calcium hydrogen phosphate, calcium acetate, calcium hydroxide, calcium lactate, calcium maleate, calcium oleate, calcium oxalate, calcium phosphate, magnesium acetate, magnesium hydrogen phosphate, magnesium phosphate, magnesium lactate, magnesium maleate, magnesium oleate, magnesium oxalate, zinc acetate, zinc hydrogen phosphate, zinc phosphate, zinc lactate, zinc maleate, zinc oleate, zinc oxalate, cysteine, methionine, d-alpha tocopherol acetate, dl-alpha tocopherol, ascorbyl palmitate, butylated hydroxyanidole, ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimefhylphenol, diterlbulylphenol, vitamin E, lecithin, ethanolamine, sucrose, lactose, dextrose, microcrystalline cellulose, silicified microcrystalline cellulose, xylitol, fructose, sorbitol, starch, poly lactic acid, polyvinyl alcohol, polyethylene glycol (including, but not limited to the following average molecular weights: 200, 300, 400, 600, 800, 1,000, 1,300-1,600, 1,450, 1,500, 2,000, 3,000, 3,000-3,700, 3,350, 4,000, 6,000, 8,000, 10,000, 12,000, 17,500, 20,000, 35,000, 40,000, 108,000, 218,000, and 511,000 Da), hydroxy propyl methyl cellulose and combinations thereof.

In various embodiments, the impermeable layer is parylene. When parylene is used as the impermeable layer, the thickness of the parylene can be one micron or more. Parylene can be a permeable or impermeable coating depending on the thickness of the polymer coating and the drug encapsulated inside the parylene coating. Generally, coatings of parylene which are thicker than one micron are considered impermeable. Coatings of parylene which are thinner than one micron can have pores, and thus creates a permeable coating. The less thick the layer of parylene, the more porous the layer, thus creating a more permeable coating, and creating a coating which is permeable to a wider range of drugs.

In various embodiments, the permeable polymer coating is selected from the group consisting of parylene, polylactic acid, polyvinyl alcohol, and combinations thereof. In particular embodiments, the permeable polymer coating is parylene.

In alternative embodiments, the method of producing a drug product comprises: providing a first compressed drug pellet and a second compressed drug pellet; joining the first drug pellet and the second drug pellet by adding a connecting substrate between the first drug pellet and the second drug pellet to produce a connected drug pellet; coating the connected drug pellet with an impermeable layer to form a coated drug pellet; cutting the coated drug pellet through the connecting substrate to form a first open end for the first drug pellet and a second open end for the second drug pellet; coating the first open end and the second open end with a permeable layer to create a release window for the first drug pellet and a release window for the second drug pellet. In various embodiments, the method further comprises compressing a first drug into the first pellet and the first drug into the second drug pellet. In various embodiments, the method further comprises compressing a first drug into the first drug pellet and a second drug into the second drug pellet.

In an embodiment, FIG. 5 depicts a first compressed drug pellet 501 and a second compressed drug pellet 502; joining the first drug pellet and the second drug pellet by adding a connecting substrate 503 between the first drug pellet and the second drug pellet to produce a connected drug pellet 504; coating the connected drug pellet with an impermeable layer to form a coated drug pellet 505; cutting the coated drug pellet through the connecting substrate to form a first open end 506 for the first drug pellet and a second open end 507 for the second drug pellet; The first open end and the second open end can be coated with a permeable layer to create a release window for the first drug pellet and a release window for the second drug pellet. In some embodiments, the first open end and the second open end do not need further coating as the connecting substrate can be used to control the release.

In various embodiments, the drug in the compressed drug pellet is a drug as discussed herein. In various embodiments, the drug is in an amount as discussed herein.

In various embodiments, the connecting substrate is selected from the group consisting of poly lactic acid, polyvinyl alcohol, polyethylene glycol (e.g., MW 3350), microcrystalline cellulose and combinations thereof.

In various embodiments, the impermeable layer is parylene. Again, as an impermeable layer, the thickness of the parylene is one micron or more.

In various embodiments, the permeable polymer coating is selected from the group consisting of parylene, polylactic acid, sugar phosphates, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene (parylene N), halogenated para-xylene (i.e. paylene C, parylene HT), β-phenol lactic acid, silicone, ethylene vinyl acetate, polyvinyl alcohol and combinations thereof. In particular embodiments, the permeable polymer coating is parylene that is one micron or less in thickness. In various embodiments, the degree of permeability (e.g., permeability to certain molecular weights) for the permeable layer can vary to suit the needs of the drug.

In various embodiments, the method of producing a drug product comprises: providing a compressed drug pellet; and coating the compressed drug pellet with a biodegradable permeable polymer. In various embodiments, the drug release is controlled by the permeability of the polymer and not the degradation of the polymer. In various embodiments, after the completion of the drug release, the polymer degrades.

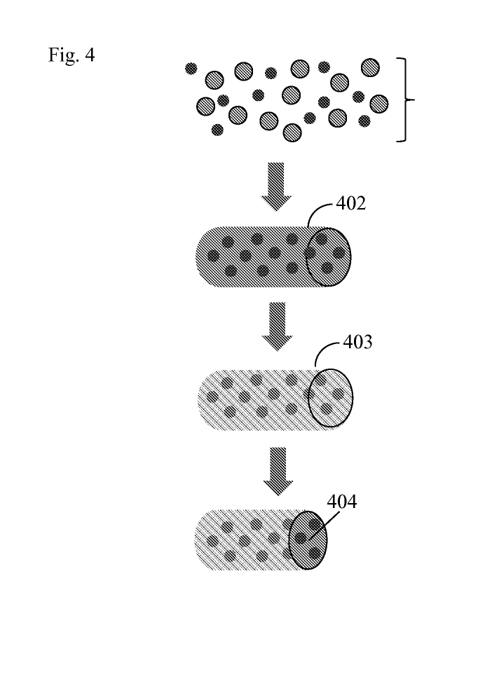

In an embodiment, FIG. 3 depicts a compressed drug pellet 301; and coating the compressed drug pellet with a biodegradable permeable polymer 302. The drug release is controlled by the permeability of the polymer and not the degradation of the polymer 303. In various embodiments, after the completion of the drug release 304, the polymer degrades 305.

In various embodiments, the drug in the compressed drug pellet is a drug as discussed herein. In various embodiments, the drug is in an amount as discussed herein.

In various embodiments, the biodegradable permeable polymer is selected from a polymer or co-polymer including at least one monomer selected from the group consisting of sugar phosphates, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, β-phenol lactic acid, and combinations thereof. In various embodiments, the degree of permeability (e.g., permeability to certain molecular weights) for the permeable layer can vary to suit the needs of the drug.

In various embodiments, the method of producing a drug product comprises using gentamicin as the drug, paralene or silicone was the impermeable casing or impermeable polymer coating, and poly lactic acid and/or parylene as the permeable polymer coating. In further embodiments, the drug product produced is used to inhibit, alleviate or treat Meniere's disease.

In various embodiments, the method of producing a drug product comprises using infliximab as the drug with hydroxy propyl methyl cellulose as the excipient, parylene or silicone as the impermeable casing or the impermeable polymer coating, and poly lactic acid and/or parylene as the permeable polymer coating. In various embodiments, the drug product is used to inhibit, alleviate or treat autoimmune inner ear disease and/or inflammation.

In various embodiments, the method of producing a drug product comprises using dexamethasone as the drug, and parylene or silicone is the impermeable casing or impermeable polymer coating, and polyvinyl alcohol and/or parylene as the permeable polymer coating. In various embodiments, the drug product is used to inhibit, alleviate, or treat inflammation, sensorineural hearing loss, autoimmune inner ear disease, noise induced hearing loss.

In various embodiments, the method of producing a drug product comprises using BDNF as the drug, and parylene or silicone as the impermeable casing or impermeable polymer coating, and poly lactic acid and/or parylene as the permeable polymer coating. In various embodiments, the drug product is used to inhibit, alleviate, or treat sensorineural hearing loss, or noise induced hearing loss.

Various embodiments of the present invention provide for a method to alleviate or treat a disease condition.

In various embodiments, the method comprises: providing a drug product of the present invention; and administering the drug product to a mammalian subject in need thereof. In various embodiments, the disease condition is Meniere's disease.

In various embodiments, the drug product according to the invention may be formulated for delivery via any route of administration. “Route of administration” may refer to any administration pathway known in the art, including but not limited to, implantation of the drug product at, in or near a desired treatment area; for example, in the middle ear near the round window, laid into mucosa of middle ear, placed into oval window, or placed into the stapes.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### In vitro release of Gentamicin

Studies were conducted in vitro to demonstrate the release of the highly soluble, gentamicin sulfate, from a packed silicone cup. A solid drug pellet (FIG. 1, 105.) was dip coated with polylactic acid and packed into a silicone cup (FIG. 1, 101.). The drug can only release from one end of the silicone and this end is subsequently coated with a controlled release polymer formulation of parylene (FIG. 1, 107.) to control its release. Two grams of parylene was loaded into the vaporization chamber to create the extended release formulation. Release was tested using a conventional dissolution system. Briefly, dissolution was performed in 2 ml distilled water in a 2 ml polypropylene microfuge tube with no stirring at room temperature. Over the course of 7 days, 40 μl gentamicin samples were taken at different time intervals, and the concentration at each time point was measured by comparing the sample absorbance against a standard curve. The gentamicin sample taken at each time point was replaced by 40 μl distilled water. Subsequent measurements were corrected for this removal of drug. Gentamicin concentration was determined as previously described. Since gentamicin does not absorb UV or visible light, a derivatizing reagent, O-phthaldialdehyde (OPA) reagent was used. Gentamicin concentration was determined by (a) mixing 40 μl gentamicin solution, 40 μl isopropanol and 40 μl OPA reagent, (b) incubation of the mixture at room temperature for 45 min, and (c) measuring UV absorbance at 333 nm.

A manufacturing process for GENTAMICIN OTIC IMPLANT (300 μg gentamicin sulfate) is manual and comprises the following (FIG. 1):

A cylindrical tube (101, 102), e.g., a silicone cup was formed from a strand of silicone tubing (Sani Tech Silicones: biopharmaceutical grade) by slicing one end with a razor blade. One end of the tube was plugged with room temperature curing silicone (Nusil MED1/2-4213). The tube was cut at the plugged end to remove excess length and create a final silicone cup of 1-2 mm in length and with an opening (103) with the following dimensions: external diameter 0.9 mm; internal diameter 0.6 mm. The final silicone cups were sterilized via autoclaving at 121° C. for 30 minutes.

A gentamicin pellet (105) was formed by placing a small amount of gentamicin sulfate drug substance into a 0.6 mm diameter mold. The gentamicin sulfate drug substance was compressed by hand using a 73 gauge (0.024 inches in diameter) metal rod (McMaster-Carr) to yield a solid gentamicin sulfate pellet.

The pellet was removed from the mold carefully by pushing out with the same metal rod. This method yielded a gentamicin sulfate pellet in a cylindrical shape with the following dimensions: diameter (D) of implant: 0.6 mm=D; length (L) of compressed gentamicin sulfate drug substance: 0.3−0.6 mm=L.

The compressed gentamicin sulfate drug substance was dip coated in polylactic acid (5% in Dichloromethane/Ethyl Acetate (2:3) solvent) and allowed to dry on a Teflon plate for 2 hours at room temperature inside a sterile biological safety cabinet.

Two or three polylactic acid coated gentamicin sulfate pellets (105) were placed inside the silicone cup, 106, using tweezers and a 73 gauge rod until the silicone cup is filled with gentamicin sulfate. The amount of gentamicin packed in each implant depends on the length of the silicone cup. This procedure was optimized to create a silicone cup of length ˜0.9 mm containing 300 μg of gentamicin drug substance.

The release window (opening) of the silicone cup containing the gentamicin pellet was coated 107 using 2 grams of parylene C dimer via a standardized vapor deposition method using a PDS 2010 Labcoter® 2 (Specialty Coating Systems).

The implants were then rinsed with 100% ethanol and allowed to dry for 30 minutes at room temperature inside a sterile biological safety cabinet.

All tools (including a 0.6 mm diameter mold, 73 gauge metal rods, tweezers, and Teflon plate) were sterilized via autoclaving at 121° C. for 30 minutes before usage.

### Example 2

### In vivo release of Gentamicin

Gentamicin sulfate releasing implants manufactured as described in Example 1 were implanted into the round window niche of young albino guinea pigs. Pharmacokinetics: Perilymph samples were collected and assayed for gentamicin sulfate (HPLC, reversed-phase, Agilent 1100 Series HPLC with AB SCIEX API 3000 MS/MS) at 1 day, 4 days, 7 days and 10 days after gentamicin implant placement.

### Example 3

### In vitro release of Immunoglobulin G

Studies have been conducted in vitro to demonstrate the release of the highly soluble, Immunoglobulin G (IgG), from a packed silicone cup. A solid drug pellet (FIG. 4, 402.) was created by mixing 75% IgG with 25% hydroxy propyl methyl cellulose (HPMC). This solid pellet was packed into a silicone cup (FIG. 1, 101.). The drug can only release from one end of the silicone and this end was subsequently coated with a controlled release polymer formulation of 5 μ1 of 10% polylactic acid (FIG. 1, 107.) to control its release. Dissolution studies were performed with IgG packed extended release formulations. For each sample, release into 600 μl water was carried out with stirring at room temperature (23° C.). Aliquots were removed at timed intervals and analyzed with UV absorption spectroscopy at 280 nm using a Spectramax Plus384 96 well plate reader. Extended release formulations of packed IgG demonstrated dissolution of drug over a ˜21 day period.

### Example 4

### In vitro release of Inf liximab

Studies were conducted in vitro to demonstrate the release of the highly soluble, Infliximab, from a packed silicone cup. A solid drug pellet (FIG. 4, 402.) was created by mixing 75% infliximab with 25% hydroxy propyl methyl cellulose (HPMC). This solid pellet was packed into a silicone cup (FIG. 1, 101.). The drug can only release from one end of the silicone and this end is subsequently coated with a controlled release polymer formulation of 5 μl of 10% polylactic acid (FIG. 1, 107.) to control its release. Dissolution studies were performed with lyophilized infliximab powder and infliximab packed extended release formulations. For each sample, release into 600 μl water was carried out with stirring at room temperature (23° C.). Aliquots were removed at timed intervals and analyzed with UV absorption spectroscopy at 280 nm using a Spectramax Plus384 96 well plate reader. Lyophilized infliximab powder releases almost immediately upon addition to solvent. Extended release formulations of packed infliximab demonstrated dissolution of drug over a ˜21 day period.

### Example 5

### In vitro release of gentamicin sulfate

The manufacturing was done as described in FIG. 2, where a drug pellet was compressed, the compressed pellet was coated with an impermeable layer of polymer (vapor deposition with parylene), one end of the coated pellet was cut to expose uncoated drug pellet, and the exposed drug was coated with a semi-permeable layer of polymer (parylene).

A manufacturing process for GENTAMICIN OTIC IMPLANT (600 μg gentamicin sulfate) is manual and comprises the following (FIG. 2):

A gentamicin pellet (201) was formed by placing a small amount of gentamicin sulfate drug substance into a mold slip fitted for a 0.9 mm diameter rod. The gentamicin sulfate drug substance was compressed by hand using a 0.9 mm diameter metal rod (McMaster-Carr) to yield a solid gentamicin sulfate pellet.

The pellet was removed from the mold carefully by pushing out with the same metal rod. This method yielded a gentamicin sulfate pellet in a cylindrical shape with the following dimensions: diameter (D) of implant: 0.9 mm=D; length (L) of compressed gentamicin sulfate drug substance: 1.0 mm=L. This yields a pellet of approximately 600 μg gentamicin sulfate.

The compressed gentamicin pellet was coated 202 using two coatings of 10 grams of parylene C dimer via a standardized vapor deposition method using a PDS 2010 Labcoter® 2 (Specialty Coating Systems). Following coating, one end of the coated pellet was cut with a scalpel to expose the end (203) for the release window.

The release window (opening) of the coated gentamicin pellet was coated 204 using 6 grams of parylene C dimer via a standardized vapor deposition method using a PDS 2010 Labcoter® 2 (Specialty Coating Systems).

Release was tested using a conventional dissolution system. Briefly, dissolution was performed in 2 ml distilled water in a 2 ml polypropylene microfuge tube with no stirring at room temperature. Over the course of 7 days, 40 μl gentamicin samples were taken at different time intervals, and the concentration at each time point was measured by comparing the sample absorbance against a standard curve. The gentamicin sample taken at each time point was replaced by 40 μl distilled water. Subsequent measurements were corrected for this removal of drug. Gentamicin concentration was determined as previously described. Since gentamicin does not absorb UV or visible light, a derivatizing reagent, O-phthaldialdehyde (OPA) reagent was used. Gentamicin concentration was determined by (a) mixing 40 μl gentamicin solution, 40 μl isopropanol and 40 μl OPA reagent, (b) incubation of the mixture at room temperature for 45 min, and (c) measuring UV absorbance at 333 nm.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method of producing a drug product, comprising:
providing an impermeable casing comprising: a sealed end, a tube, and an open end;
placing one or more compressed drug pellets into the impermeable casing; and
coating the open end with a permeable polymer coating to produce a release window to control the release of the drug.

2. The method of claim 1, wherein the tube comprises a polymer or a co-polymer comprising at least one monomer selected from the group consisting of a sugar phosphate, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene, halogenated para-xylene, β-phenol lactic acid, silicone, ethylene vinyl acetate, polyvinyl alcohol and combinations thereof.

3. The method of claim 1, wherein the impermeable casing comprises parylene.

4. The method of claim 1, wherein the drug is selected from the group consisting of anti-inflammatory agent, analgesic agent, corticosteroid, growth factor, antioxidant, TNF-α inhibitor, volume expanding agent, vasodilating agent, antihistaminic agent, anticholinergic agent, antibiotic agent, antiviral agent, immunosuppressive agent, diuretic agent, antacid, H2-blocker, antiemetic, calcium channel blocker, anticancer agent, vitamin, vascular rheologic agent, neuroprotective agent, neuromodulator, and anti-apoptotic agent.

5. The method of claim 1, wherein the drug is gentamicin sulfate, immunoglobulin G, or infliximab.

6. The method of claim 1, wherein the drug is fentanyl citrate, aspirin, salicylate, ibuprofen, naproxen, droperidol, prochlorperazine, dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, adalimumab, batahistine, niacin, papaverine, meclizine, dimenhydrinate, scopolamene, promethazine, glycopyrrolate, propantheline, atropine, ampicillin, cefuroxime, ceftriaxone, ciprofloxacin, finafloxacin, gatifloxacin, levofloxacin, moxifloxacin, ofloxacin, gentamicin, tobramycin, clindamycin, amoxicillin, cyclophosphamide, cyclosporine, thiazide, triamterene, nizatidine, cimetidine, metoclopramide, diphenidol, diltiazem, nifedipine, or verapamil.

7. (canceled)

8. (canceled)

9. The method of claim 1, wherein the drug is dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, or adalimumab.

10. The method of claim 1, wherein the permeable polymer coating is 5 nanometers to 50 microns thick, and the permeable polymer is a polymer or a co-polymer comprising at least one monomer selected from the group consisting of a sugar phosphate, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene, halogenated para-xylene, β-phenol lactic acid, silicone, ethylene vinyl acetate, polyvinyl alcohol and combinations thereof.

11. The method of claim 1, wherein the permeable polymer coating is 5 nanometers to 50 microns thick, and the permeable polymer is selected from the group consisting of parylene, polylactic acid, polyvinyl alcohol and combinations thereof.

12. The method of claim 1, wherein the permeable polymer coating is less than one micron thick and the permeable polymer is parylene.

13. A method of producing a drug product, comprising:
providing one or more compressed drug pellets;
depositing an impermeable coating layer on the one or more drug pellets to produce a coated drug pellet;
cutting a first end of the of the coated drug pellet to create an open end; and
coating the open end with a permeable layer of a polymer to create a release window.

14. The method of claim 13, wherein the compressed drug pellet comprise a drug, a polymer and/or an excipient.

15. The method of claim 13, wherein two or more drug pellets are provided and the method further comprises joining the two or more drug pellets with a connecting substrate between the two or more drug pellets to produce a connected drug pellet.

16. The method of claim 15, wherein the connecting substrate is selected from the group consisting of poly lactic acid, polyvinyl alcohol, polyethylene glycol, microcrystalline cellulose and combinations thereof.

17. The method of claim 15, wherein cutting the first end comprises cutting the connected drug pellet through the connecting substrate to form a first open end on a first coated drug pellet and a second open end on the second coated drug pellet.

18. The method of claims 13, wherein the impermeable coating layer comprises a polymer or a co-polymer comprising at least one monomer selected from the group consisting of a sugar phosphate, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene, halogenated para-xylene, β-phenol lactic acid, silicone, ethylene vinyl acetate, polyvinyl alcohol and combinations thereof

19. The method of claim 13, wherein the impermeable coating layer comprises parylene and is one micron or more in thickness.

20. The method of claim 13, wherein the drug is selected from the group consisting of anti-inflammatory agent, analgesic agent, corticosteroid, growth factor, antioxidant, TNF-α inhibitor, volume expanding agent, vasodilating agent, antihistaminic agent, anticholinergic agent, antibiotic agent, antiviral agent, immunosuppressive agent, diuretic agent, antacid, H2-blocker, antiemetic, calcium channel blocker, anticancer agent, vitamin, vascular rheologic agent, neuroprotective agent, neuromodulator, and anti-apoptotic agent.

21. The method of claim 13, wherein the drug is gentamicin sulfate immunoglobulin G. or infliximab.

22. The method of claim 13, wherein the drug is fentanyl citrate, aspirin, salicylate, ibuprofen, naproxen, droperidol, prochlorperazine, dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, adalimumab, batahistine, niacin, papaverine, meclizine, dimenhydrinate, scopolamene, promethazine, glycopyrrolate, propantheline, atropine, ampicillin, cefuroxime, ceftriaxone, ciprofloxacin, finafloxacin, gatifloxacin, levofloxacin, moxifloxacin, ofloxacin, gentamicin, tobramycin, clindamycin, amoxicillin, cyclophosphamide, cyclosporine, thiazide, triamterene, nizatidine, cimetidine, metoclopramide, diphenidol, diltiazem, nifedipine, or verapamil.

23. (canceled)

24. (canceled)

25. The method of claim 13, wherein the drug is dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, or adalimumab.

26. The method of claim 13 or 17, wherein the permeable layer of a polymer is 5 nanometers to 50 microns thick, and the permeable polymer is a polymer or a co-polymer comprising at least one monomer selected from the group consisting of a sugar phosphate, alkylcellulose, hydroxyalkylcelluloses, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, para-xylene, halogenated para-xylene, β-phenol lactic acid, silicone, ethylene vinyl acetate, polyvinyl alcohol and combinations thereof.

27. The method of claim 13, wherein the permeable polymer coating is 5 nanometers to 50 microns thick, and the permeable polymer is selected from the group consisting of parylene, polylactic acid, polyvinyl alcohol and combinations thereof.

28. The method of claim 13, wherein the permeable polymer layer is less than one micron thick and the permeable polymer is parylene.

29. A method of producing a drug product, comprising:
providing a compressed drug pellet; and
coating the compressed drug pellet with a biodegradable permeable polymer.

30. The method of claim 29, wherein the drug is selected from the group consisting of anti-inflammatory agent, analgesic agent, corticosteroid, growth factor, antioxidant, TNF-α inhibitor, volume expanding agent, vasodilating agent, antihistaminic agent, anticholinergic agent, antibiotic agent, antiviral agent, immunosuppressive agent, diuretic agent, antacid, H2-blocker, antiemetic, calcium channel blocker, anticancer agent, vitamin, vascular rheologic agent, neuroprotective agent, neuromodulator, and anti-apoptotic agent.

31. The method of claim 29, wherein the drug is fentanyl citrate, aspirin, salicylate, ibuprofen, naproxen, droperidol, prochlorperazine, dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, adalimumab, batahistine, niacin, papaverine, meclizine, dimenhydrinate, scopolamene, promethazine, glycopyrrolate, propantheline, atropine, ampicillin, cefuroxime, ceftriaxone, ciprofloxacin, finafloxacin, gatifloxacin, levofloxacin, moxifloxacin, ofloxacin, gentamicin, tobramycin, clindamycin, amoxicillin, cyclophosphamide, cyclosporine, thiazide, triamterene, nizatidine, cimetidine, metoclopramide, diphenidol, diltiazem, nifedipine, or verapamil.

32. The method of claim 29, wherein the drug is immunoglobulin G or infliximab.

33. (canceled)

34. The method of claim 29, wherein the drug is dexamethasone, dexamethasone phosphate, dexamethasone acetate, hydrocortisone, fluticasone proprionate, flusinolone, beclomethasone, triamcinalone, prednisone, prednisolone, methylprednisolone, triamcinolone, IGF-1, FGF-2, BDNF, reduced glutathione, N-methyl-(D)-glucaminedithiocarbamate, (D)-methionine, infliximab, etanercept, or adalimumab.

35. The method of claim 29, wherein the biodegradable permeable polymer is selected from a polymer or co-polymer including at least one monomer selected from the group consisting of sugar phosphates, lactic acid, glycolic acid, β-propiolactone, β-butyrolactone, γ-butyrolactone, pivalolactone, α-hydroxy butyric acid, α-hydroxyethyl butyric acid, α-hydroxy isovaleric acid, α-hydroxy-β-methyl valeric acid, α-hydroxy caproic acid, α-hydroxy isocaproic acid, α-hydroxy heptanic acid, α-hydroxy octanic acid, α-hydroxy decanoic acid, α-hydroxy myristic acid, α-hydroxy stearic acid, α-hydroxy lignoceric acid, β-phenol lactic acid, and combinations thereof.

36. A method of inhibiting, alleviating or treating a disease condition, comprising:
providing a drug product of claim 1; and
administering the drug product to a mammalian subject in need thereof.

37. (canceled)

38. (canceled)

39. A method of inhibiting, alleviating or treating a disease condition, comprising:
providing a drug product of claim 13; and
administering the drug product to a mammalian subject in need thereof

40. (canceled)

41. (canceled)

42. A method of inhibiting alleviating or treating a disease condition, comprising:
providing a drug product of claim 29; and
administering the drug product to a mammalian subject in need thereof

43. (canceled)

44. (canceled)
